(19) United States
(12) Patent Application Publication
(10) Pub. No.: US 2003/0236202 A1
(43) Pub. Date: Dec. 25, 2003

(54) Natural products and derivatives thereof for protection against neurodegenerative diseases

(76) Inventors: Arjan Geelings, Granada (ES); Eduardo Lopez-Huertas Leon, Granada (ES); Juan Carlos Morales Sanchez, Granada (ES); Julio Boza Puerta, Granada (ES); Jesus Jimenez Lopez, Granada (ES)

Correspondence Address: KATTEN MUCHIN ZAVIS ROSENMAN, 575 MADISON AVENUE, NEW YORK, NY, 10022-2585, US

(21) Appl. No.: 10/406,791

(22) Filed: Apr. 3, 2003

(30) Foreign Application Priority Data
Apr. 3, 2002 (WO) PCT/EP02/03675

(51) Int. Cl. A61K 31/7024; A61K 31/23; C07C 69/88; C07H 15/04

(52) U.S. Cl. 514/25; 514/568; 514/734; 536/116; 560/68; 562/476; 554/229; 514/548

(57) ABSTRACT

The invention relates to the use of phenolic compounds and derivatives thereof for protection against neurodegenerative diseases, to compositions comprising these compounds, and to some novel phenolic compounds.

### FIELD OF THE INVENTION

The present invention relates to the use of phenolic compounds and derivatives thereof for protection against neurodegenerative diseases, to compositions comprising these compounds, and to some novel phenolic compounds.

### BACKGROUND OF THE INVENTION

During life, cells in all biological systems are exposed to oxidation. Oxidation leads to the formation of free radicals, which are compounds that contain one, or more unpaired electrons. These radicals react easily with other molecules in the cell, and during life the continuous attack of free radicals may alter cellular mechanisms and ultimately may lead to cell death. Free radicals and molecules that generate them are often classified as reactive oxygen species (ROS) and cells have developed mechanisms to cope with these compounds. However, problems may arise when these mechanisms are not sufficient to eliminate all ROS present. Under these conditions oxidative stress may cause cellular damage and subsequent cellular death, mainly by apoptosis. One of the principle factors that reduce ROS and oxidative stress are antioxidants. Cells produce their own antioxidants (e.g. glutathione) but may also acquire dietary antioxidants.

The brain is particularly vulnerable to oxidative stress for several reasons. First of all, because of its high-energy demand, the brain consumes 20% of all oxygen used. The consumption of oxygen leads to the production of ROS and therefore the brain generates high amounts of these reactive oxygen species. Additionally, excitatory amino acids (e.g. glutamate) generate high levels of ROS on their massive release after brain injury. Glutathione is the main antioxidant in brain. After 30 years of age, the concentration of this antioxidant in human brain declines. Therefore, it is believed that during aging oxidative stress becomes more abundant. In addition, due to the blood brain barrier, many dietary antioxidants (like vitamin C, carotenoids and flavonoids) are unable to enter the brain.

Certain diseases of the brain and nervous system are thought to involve free radical processes and oxidative damage, either as a primary cause or as a consequence of the disease. Examples of brain disease where oxidative stress plays an important role are Alzheimer disease, cognitive dysfunction (loss of memory), and Parkinson disease. For the treatment and prevention of these diseases, antioxidants might be useful. Since most dietary antioxidants cannot pass the blood brain barrier, there is a need for antioxidants that penetrate this biological barrier (Gilgun-Sherki et al. Neuropharmacology 2001, 40(8), 959-75). The use of antioxidants, however, is already known in the art in the prevention of cardiovascular diseases (WO 97/0667 and WO 01/45514).

On the other hand, several studies have demonstrated that copper (Cu2), zinc (Zn²⁺) and iron (Fe³⁺) ions play an important role in Alzheimer's and Parkinson's disease. Concentrations of these ions are elevated in beta-amyloid plaques and in Lewy bodies, the beta-amyloid analogue in Parkinson's disease. Moreover, these metal ions may initiate the aggregation process of beta-amyloid peptides leading to insoluble fibril formation. This has led some investigators to study the effect of metal chelators on the formation of beta-amyloid plaques. The results of in vivo studies demonstrated that a zinc and copper chelator reduced amyloid deposits in a mouse model for Alzheimer's disease (Cherny et al. Neuron 2001, 30(3), 665-76). Therefore, metal chelators are thought to have neuroprotective properties.

Basic and clinical researchers have been investigating neurodegenerative disease, and how to protect neurons from dying, a principal hallmark of these diseases. Since oxidative stress is a common theme in many neurodegenerative diseases and may lead to neuronal death, some antioxidants have been investigated for their potential to reduce oxidative stress. Cases of partial improvement have been reported, but to date no potent antioxidants have been found that fulfil the basic requirements for their use in the treatment of neurodegenerative disease; which are that they need to:

1. enter the brain in sufficient amounts

2. have antioxidant activity once inside the central nervous system, and

3. be non-toxic

Moreover, it would be advantageous if the antioxidant is a natural product known for its presence in dietary products. Most antioxidants present in food however do not fulfil the first requirement since they are unable to pass the blood brain barrier.

Another important aspect of many neurodegenerative diseases is the accumulation of insoluble protein aggregates (e.g. beta-amyloid plaques in Alzheimer's disease and Lewy bodies in Parkinson's disease). The exact mechanism of how these aggregates are formed is still not completely understood, but metal ions are thought to play an important role. For instance, zinc and copper ions in low concentrations facilitate the formation of beta-amyloid plaques, and iron, among other metal ions, is found in high concentrations in Lewy bodies. Chelators of these metal ions are known to dissolve above-mentioned aggregates and therefore are useful as neuroprotectants in neurodegenerative disease. To be useful in neuroprotection, chelators of metal ions need to fulfil three requirements. tTey need to:

1. readily enter the brain in sufficient amounts

2. solubilize protein aggregates in vivo, and

3. be non-toxic.

In this invention we describe a natural product isolated from olives (2-(3,4-dihydroxyphenyl) ethanol, hydroxytyrosol) that combines all above-mentioned requirements for its use in a treatment against neurodegenerative disease. This compound readily enters the brain; it is a potent antioxidant even when it is localized in the brain, and it chelates metal ions. Moreover, hydroxytyrosol is a potent inhibitor of monoamine oxidase (MAO-B). This combination of characteristics makes hydroxytyrosol suitable for the treatment of Alzheimer's, Parkinson's and other neurodegenerative diseases. Moreover, this compound is a natural product and can be isolated from olives. Therefore, this compound may be used as a dietary supplement or in pharmaceutical preparations. We also describe a series of hydroxytyrosol esters that, upon hydrolysis, for instance in the stomach of a consumer, liberate hydroxytyrosol. These esters are very useful for the conservation of hydroxytyrosol, because in the ester form, hydroxytyrosol is prevented from oxidation. Another advantage of using hydroxytyrosol esters is their lipid solubility. Finally, we describe another natural occurring phenolic compound, tyrosol (2-(4-hydroxyphenyl) ethanol), which is a powerful antioxidant and readily enters the nervous system. This compound is also useful in the treatment of neurodegenerative diseases where oxidative stress plays an important role.

### SUMMARY OF THE INVENTION

The present invention provides, therefore, phenolic compounds and derivatives thereof for protection against neurodegenerative diseases, compositions comprising these compounds and some novel phenolic compounds.

An aspect of the invention relates to phenolic compounds and derivatives thereof for the treatment or prevention of neurodegenerative diseases such as: Creutzfeldt-Jakob disease, Alzheimer's disease, Huntington's disease, Lewy body disease, Parkinson's disease, Pick's disease, amyotrophic lateral sclerosis, neurofibromatosis, brain injury, stroke, multiple sclerosis, loss of memory, or multiple infarct dementia.

A second aspect of the invention relates to some novel phenolic compounds and their derivatives.

Finally, a third aspect of the invention relates to compositions comprising phenolic compounds and derivatives thereof for the treatment or prevention of neurodegenerative diseases.

### DETAILED DESCRIPTION OF THE INVENTION

The first aspect of the invention relates to phenolic compounds and derivatives thereof for the treatment or prevention of neurodegenerative diseases,

Said diseases are, for instance, such as Creutzfeldt-Jakob disease, Alzheimer's disease, Huntington's disease, Lewy body disease, Parkinson's disease, Pick's disease, amyotrophic lateral sclerosis, neurofibromatosis, brain injury, stroke, multiple sclerosis, loss of memory, or multiple infarct dementia.

Two phenolic compounds provided by the invention, hydroxytyrosol and tyrosol, are both capable of entering the brain in relatively large quantities and act as antioxidants. Furthermore, hydroxytyrosol acts as an inhibitor of MAO-B and as a chelator of iron, zinc and copper ions. Because of these characteristics both phenolic compounds function as neuroprotectants in the therapy of neurodegenerative diseases.

The object of this invention is to increase the antioxidant levels in brain and therefore to reduce the oxidative stress in this tissue. A second objective of this invention is to reduce the concentrations of iron, zinc and copper ions in brain, and by that means inhibit the formation of protein precipitates. Hydroxytyrosol, tyrosol, and compounds that liberate these phenolics after their hydrolysis can be administered as a dietary supplement or as a pharmaceutical preparation and may, thus, be used in the treatment of diseases where oxidative stress, insoluble protein deposits, and subsequent neuronal loss play an important role.

Phenolic compounds can be found among others in olives, red wine, black tea, and in a wide variety of fruits and vegetables. One of the major phenolic compounds from olives is hydroxytyrosol, which is a very powerful antioxidant. It can be isolated from the leaf of the olive tree but is also found in the olive fruit and in olive oil. The health benefits of the Mediterranean diet are partly due to the consumption of olive oil and the presence of phenolic compounds therein.

Our studies showed that, when taken orally, hydroxytyrosol is rapidly taken up from the intestinal tract and subsequently is able to pass the blood brain barrier in relatively large amounts. For this reason this compound accumulates in the cerebrospinal fluid and from there it has access to the cells in the central nervous system. Interestingly, cerebrospinal fluid taken from rats after oral administration of hydroxytyrosol showed a considerable increase in total antioxidant capacity. This makes hydroxytyrosol very useful in the treatment of neurological diseases where oxidative stress plays an important role. Different kinds of stressors may induce oxidative stress and oxidative damage that leads to cell death. In Alzheimer's disease, the beta-amyloid peptide accumulates in brain tissue. This accumulation may lead to oxidative stress, and large amounts of cells die leading to the typical symptoms associated to Alzheimer's disease. Glutamate is the major excitatory neurotransmitter in the brain, but is also a powerful oxidant. Furthermore, different mechanisms may induce the production of hydrogen peroxide which is a very potent prooxidant in cells. We found that hydroxytyrosol protects cells of the central nervous system from dying after exposure to these stressors (beta-amyloid, glutamate and hydrogen peroxide). This, together with the fact that hydroxytyrosol easily enters the brain, makes it a powerful neuroprotectant. Since oxidative stress plays an important role in many neurodegenerative diseases and consequently large amounts of cells die, this phenolic compound is especially useful in the treatment of these diseases.

Besides its antioxidant capacity, we found that hydroxytyrosol is a chelator of iron, zinc and copper ions. Since zinc, copper and iron ions induce the formation of beta-amyloid plaques, chelators of these ions might solubilize these plaques. This is thought to be beneficial for Alzheimer disease therapy (Cherny et al. Neuron 2001, 30(3), 665-76). The hydroxytyrosol is not only is a strong chelator of iron, zinc and copper ions, hydroxytyrosol does indeed solubilize beta-amyloid deposits. Therefore, hydroxytyrosol has a dual neuroprotective action. It is a potent antioxidant and reduces iron, zinc and copper concentrations in certain brain regions. Both actions are beneficial in neurodegenerative disease, and so this phenolic compound is advantageous over compounds that are solely antioxidants or chelators of metal ions.

In Parkinson's disease large amounts of dopaminergic neurons die in the substantia nigra. Dopamine replacement therapy, in the form of levodopa supplementation, has been used successfully in the treatment of Parkinson's disease. Inhibition of dopamine metabolism is a valuable adjunct to exogenous dopaminergic replacement. Therefore, inhibitors of MAO-B have been used to treat early and advanced Parkinson disease for a number of years. Although some controversy remains, existing evidence raises the possibility that MAO-B inhibition may confer a protective effect in Parkinson's disease, delaying the progression of the underlying pathology. We found that hydroxytyrosol is a strong inhibitor of MAO-B. Therefore, this activity of hydroxytyrosol may be of additional use in the treatment of this disease.

Tyrosol is a second neuroprotector included within the scope of this invention is. Tyrosol is also a phenolic compound found in olives. As with hydroxytyrosol, tyrosol is a powerful antioxidant and is rapidly taken up from the intestinal tract. Moreover, it is capable of passing the blood brain barrier in relatively large amounts. We detected tyrosol in cerebrospinal fluid of rats which had received tyrosol orally. Therefore, tyrosol is very useful in the treatment of neurodegenerative diseases where oxidative stress plays an important role. Since tyrosol increases the total antioxidant levels of the brain, it can be used to treat disorders like Alzheimer's and Parkinson's disease. It can also be used to treat stroke and loss of memory.

This invention also provides hydroxytyrosol and tyrosol esters for the treatment or prevention of neurodegenerative diseases. These esters have several advantages over hydroxytyrosol and tyrosol. They are more lipid soluble and the hydroxyl groups of hydroxytyrosol and tyrosol are protected from oxidation by a fatty acid ester. The ester bond is rapidly hydrolysed in an acid environment liberating the fatty acid and hydroxytyrosol or tyrosol.

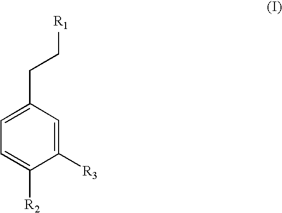

The hydroxytyrosol and tyrosol fatty acid esters described in this invention have a common structure as represented by formula 1. Within this structure groups R1, R2, and R3 vary leading to a series of related hydroxytyrosol or tyrosol derivatives. Groups R1 and R2 can be either a hydroxyl group or a hydroxyl group protected with a fatty acid chain via an ester bond. Group R3 can be either a hydrogen (in the case of tyrosol or tyrosol esters), a hydroxyl group (in the case of hydroxytyrosol or hydroxytyrosol esters) or a hydroxyl group protected with a fatty acid chain via an ester bond (in the case of hydroxytyrosol esters). Each compound must contain one, two or three fatty acid chains with a length from C2 to C22. Therefore, R₁ and R₂ are independently selected from: OH, OCOalkyl or OCOalkenyl, and R₃ is either H, OH, OCOalkyl or OCOalkenyl, and wherein the alkyl or alkenyl group contains from 2 to 22 carbon atoms.

The novel hydroxytyrosol and tyrosol esters form the second aspect of the invention. They include the phenolic compounds (2), (3), (4), (5), (9), (10), (12), (13), (14), (15), (17), (18), (19), and (20) represented by the formulae VI, VII, VIII, IX, XIII, XIV, XVI, XVII, XVIII, XIX, XXI, XXII, XXIII, XIV, respectively. Whereas compounds (1), (6) and (7) are natural products, compounds (8), (11) and (16) have not been found in nature. Compounds (1) to (20) have been synthesised following standard esterification methods. The synthesis of some of these compounds, namely the compounds of formulae V, VII, XI, XV and XVII, are exemplified below.

Illustrations of some hydroxytyrosol and tyrosol derivatives included in this invention are:

(1) 2-(3,4-Dihydroxyphenyl) ethyl acetate, an hydroxytyrosol derivative having the formula V, wherein R2 and R3 are hydroxyl groups, and R1 is an hydroxyl group protected with an acetic acid chain via an ester bond.

(2) 2-(3,4-Dihydroxyphenyl) ethyl oleate, an hydroxytyrosol derivative having the formula VI, wherein R2 and R3 are hydroxyl groups, and R1 is an hydroxyl group protected with an oleic acid chain via an ester bond.

(3) 2-(3,4-Dihydroxyphenyl) ethyl stearate, an hydroxytyrosol derivative having the formula VII, wherein R1 and R2 are hydroxyl groups, and R3 is an hydroxyl group protected with a stearic acid chain via an ester bond.

(4) 2-(3,4-Dihydroxyphenyl) ethyl docosahexaenoate, an hydroxytyrosol derivative having the formula VIII, wherein R1 and R2 are hydroxyl groups, and R3 is an hydroxyl group protected with a docosahexaenoic acid chain via an ester bond.

(5) 2-(3,4-Dihydroxyphenyl) ethyl eicosapentaenoate, an hydroxytyrosol derivative having the formula IX, wherein R1 and R2 are hydroxyl groups, and R3 is an hydroxyl group protected with an eicosapentaenoic acid chain via an ester bond.

(6) 2-(4-Hydroxyphenyl) ethyl acetate, a tyrosol derivative having the formula X, wherein R2 is an hydroxyl group, R3 is an hydrogen, and R1 is an hydroxyl group protected with an acetic acid chain via an ester bond.

(7) 2-(4-Hydroxyphenyl) ethyl oleate, a tyrosol derivative having the formula XI, wherein R2 is an hydroxyl group, R3 is an hydrogen, and R1 is an hydroxyl group protected with an oleic acid chain via an ester bond.

(8) 2-(4-Hydroxyphenyl) ethyl stearate, a tyrosol derivative having the formula XII, wherein R2 is an hydroxyl group, R3 is an hydrogen, and R1 is an hydroxyl group protected with a stearic acid chain via an ester bond.

(9) 2-(4-Hydroxyphenyl) ethyl docosahexaenoate, a tyrosol derivative having the formula XIII, wherein R2 is an hydroxyl group, R3 is an hydrogen, and R1 is an hydroxyl group protected with a docosahexaenoic acid chain via an ester bond.

(10) 2-(4-Hydroxyphenyl) ethyl eicosapentaenoate, a tyrosol derivative having the formula XIV, wherein R2 is an hydroxyl group, R3 is an hydrogen, and R1 is an hydroxyl group protected with an eicosapentaenoic acid chain via an ester bond.

(11) 2-(3,4-Diacetoxyphenyl) ethyl acetate, an hydroxytyrosol derivative having the formula XV, wherein R1, R2, and R3 are hydroxyl groups protected with an acetic acid chain via an ester bond.

(12) 2-(3,4-Dioleyloxyphenyl) ethyl oleate, an hydroxytyrosol derivative having the formula XVI, wherein R1, R2, and R3 are hydroxyl groups protected with an oleic acid chain via an ester bond.

(13) 2-(3,4-Distearyloxyphenyl) ethyl stearate, an hydroxytyrosol derivative having the formula XVII, wherein R1, R2, and R3 are hydroxyl groups protected with a stearic acid chain via an ester bond.

(14) 2-(3,4-Didocosahexaenoyloxyphenyl) ethyl docosahexaenoate, an hydroxytyrosol derivative having the formula XVIII, wherein R1, R2, and R3 are hydroxyl groups protected with a docosahexaenoic acid chain via an ester bond.

(15) 2-(3,4-Dieicosapentaenoyloxyphenyl) ethyl eicosapentaenoate, an hydroxytyrosol derivative having the formula XIX, wherein R1, R2, and R3 are hydroxyl groups protected with an eicosapentaenoic acid chain via an ester bond.

(16) 2-(4-Acetoxyphenyl) ethyl acetate, a tyrosol derivative having the formula XX, wherein R3 is an hydrogen, and wherein R1 and R2 are hydroxyl groups protected with an acetic acid chain via an ester bond.

(17) 2-(4-Oleyloxyphenyl) ethyl oleate, a tyrosol derivative having the formula XXI, wherein R3 is an hydrogen, and wherein R1 and R2 are hydroxyl groups protected with an oleic acid chain via an ester bond.

(18) 2-(4-Stearyloxyphenyl) ethyl stearate, a tyrosol derivative having the formula XXII, wherein R3 is an hydrogen, and wherein R1 and R2 are hydroxyl groups protected with a stearic acid chain via an ester bond.

(19) 2-(4-Docosahexaenoyloxyphenyl) ethyl docosahexaenoate, a tyrosol derivative having the formula XXIII, wherein R3 is an hydrogen, and wherein R1 and R2 are hydroxyl groups protected with a docosahexaenoic acid chain via an ester bond.

(20) 2-(4-Eicosapentaenoyloxyphenyl) ethyl eicosapentaenoate, a tyrosol derivative having the formula XXIV, wherein R3 is an hydrogen, and wherein R1 and R2 are hydroxyl groups protected with a eicosapentaenoic acid chain via an ester bond.

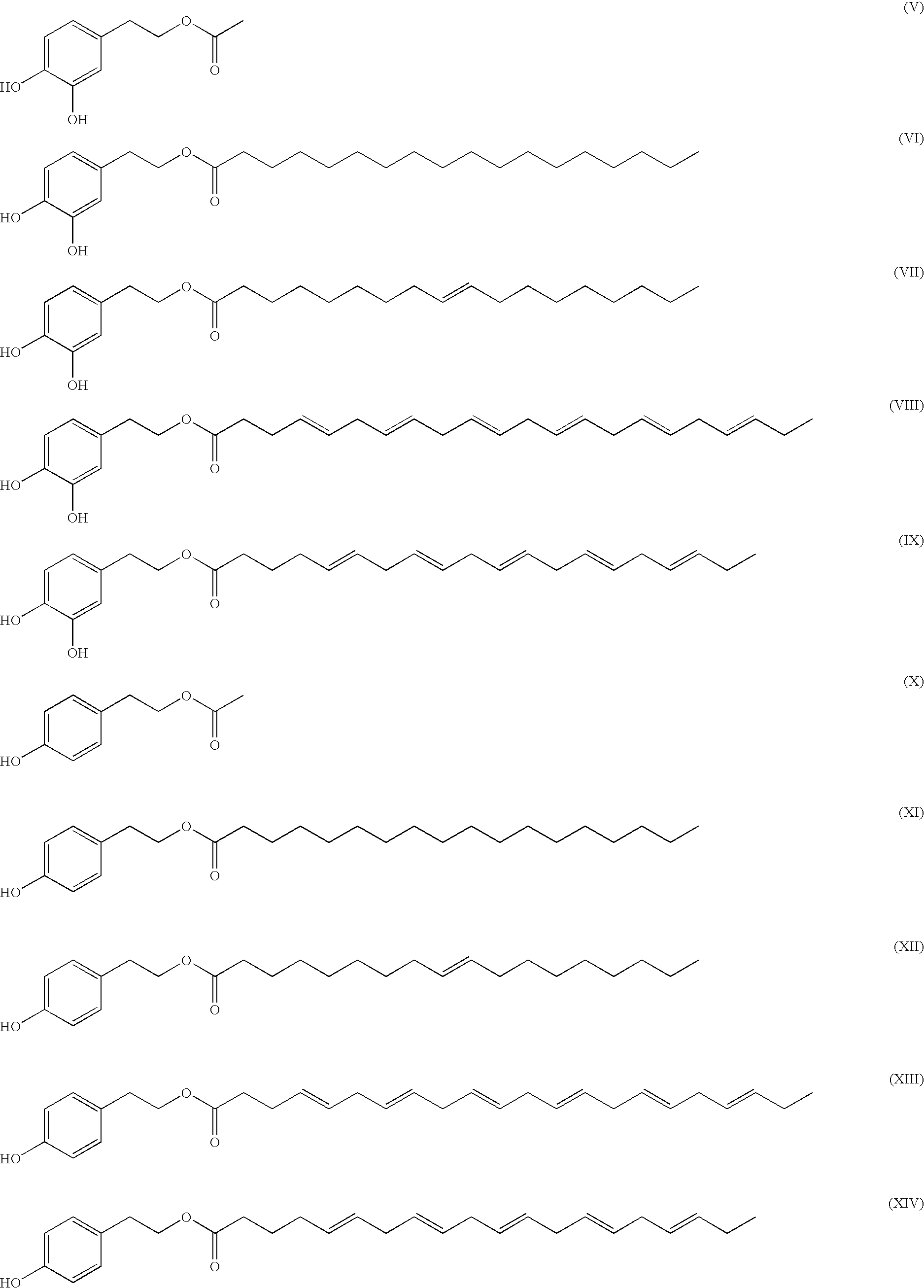

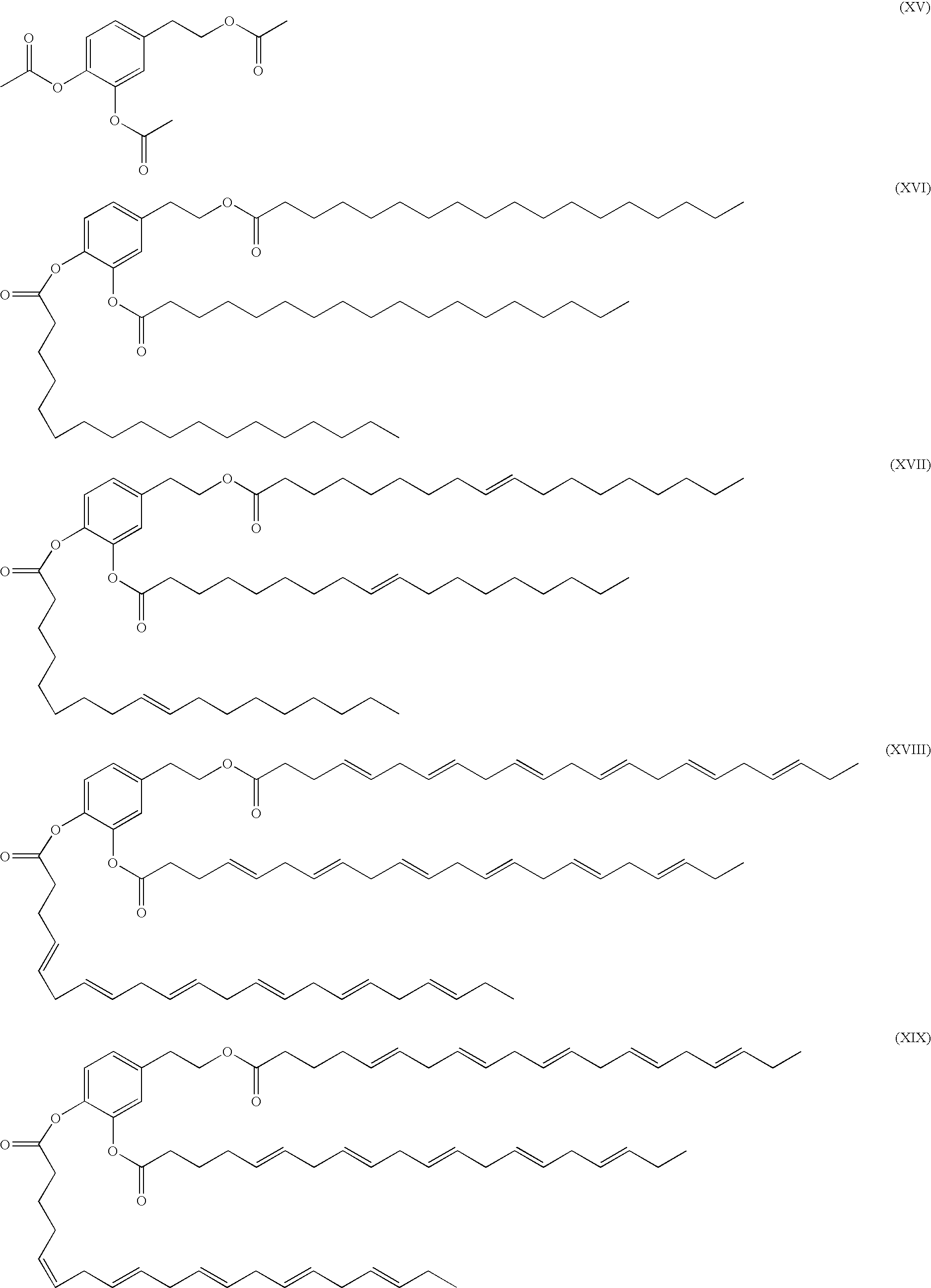

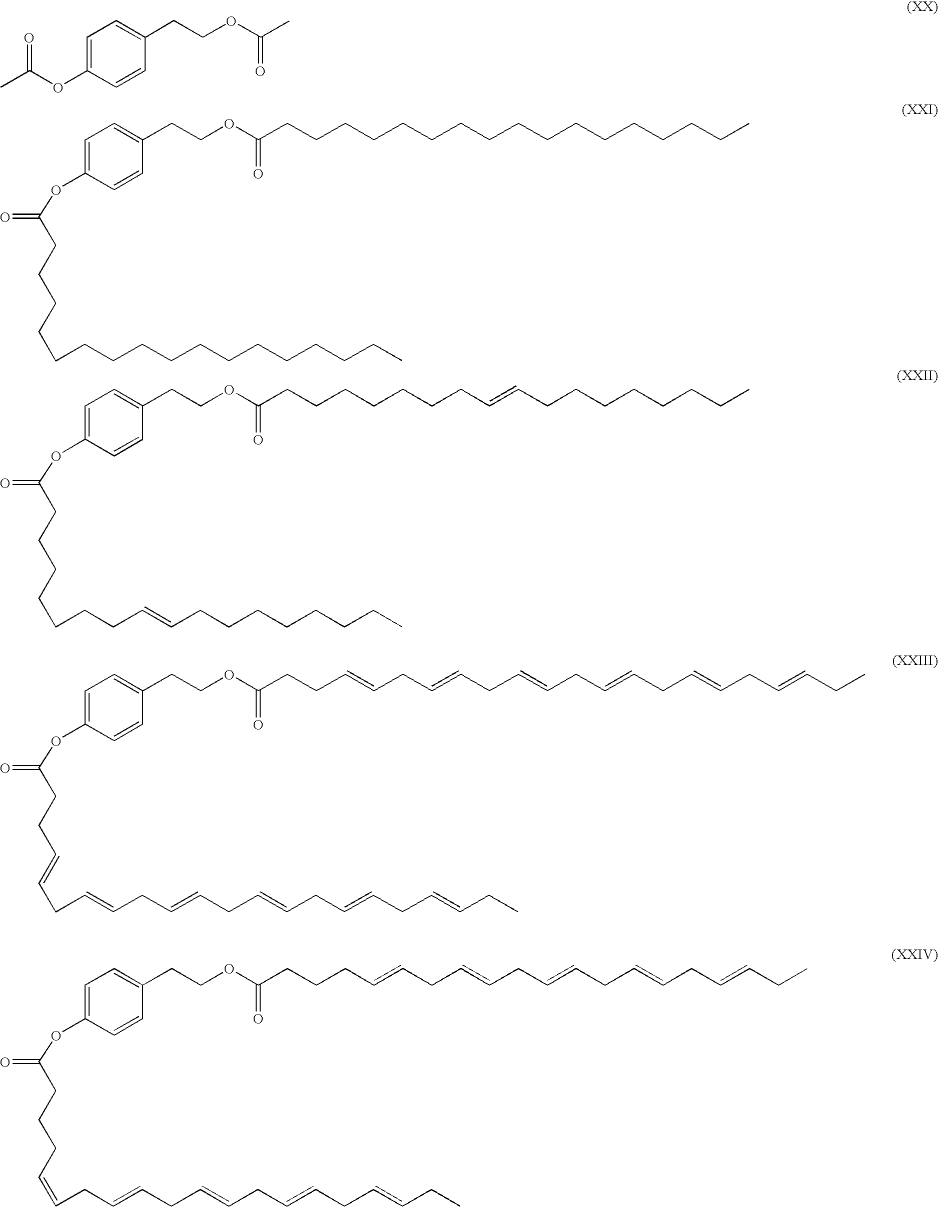

Hydroxytyrosol is represented by formula II, where R1, R2 and R3 are hydroxyl groups, and tyrosol is presented by formula III, wherein R1 and R2 are hydroxyl groups and R3 is an hydrogen.

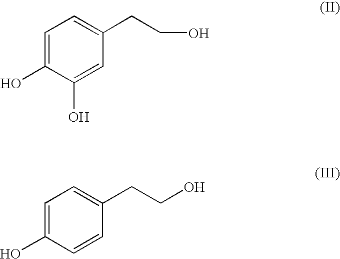

Hydroxytyrosol and tyrosol are sensitive to oxidation and are hydrophilic. This last characteristic might be problematic if hydroxytyrosol or tyrosol are intended to be used in fat-based food products. Oxidation of hydroxytyrosol and tyrosol clearly affect the stability and preservation of both compounds. The hydroxytyrosol and tyrosol derivatives presented in this invention avoid these two problems. The hydroxyl groups on these derivatives are protected from oxidation by preparing hydroxytyrosol or tyrosol fatty acid esters. When compared to hydroxytyrosol and tyrosol, the hydroxytyrosol and tyrosol fatty acid esters are much more resistant against oxidation. At the same time, depending on the length of the fatty acid chain of the fatty acid esters, their solubility in fat-based food products will be increased.

Hydroxytyrosol or tyrosol esters with a wide range of solubilities can be prepared, from totally water-soluble hydroxytyrosol or tyrosol derivatives when acetic acid is used in the formation of the ester to totally oil-soluble hydroxytyrosol or tyrosol derivatives when oleic acid is used in the formation of the ester. The hydroxytyrosol and tyrosol derivatives are hydrolysed in the intestinal tract of rats to their two components, hydroxytyrosol or tyrosol and the fatty acid. Hydroxytyrosol or tyrosol are then rapidly absorbed, being detected in plasma and cerebrospinal fluid. This implies that, after their hydrolysis, the fatty acid esters can act as antioxidants to prevent diseases with an oxidative stress origin.

Oleuropein (formula IV) is another compound included in this invention. Oleuropein is a natural product found in olives, and like the fatty acid esters mentioned above, liberates hydroxytyrosol after its hydrolysis. Therefore, oleuropein is protected from oxidation, and because it liberates hydroxytyrosol in the stomach of a consumer, it is also useful in the treatment and prevention of neurodegenerative disease.

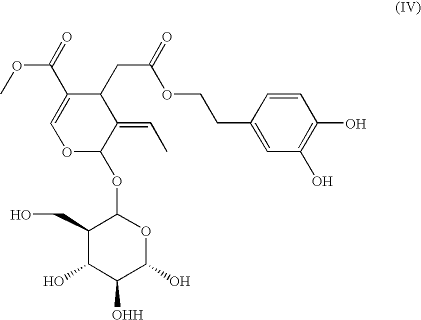

The third aspect of the invention relates to compositions comprising phenolic compounds or derivatives thereof for the treatment of neurodegenerative diseases. Said compositions may comprise hydroxytyrosol, tyrosol, esters of hydroxytyrosol, esters of tyrosol or combinations thereof, and may be pharmaceutical compositions or food compositions.

The pharmaceutical compositions of the invention may contain one or more appropriate binders, carriers and/or further auxiliary materials. The carrier materials, binders and/or auxiliary materials must be pharmaceutically and pharmacologically tolerable, so that they can be combined with the other components of the formulation or preparation and do not exert adverse effects on the organism treated.

The formulations include those, which are suitable for oral or parenteral (including subcutaneous, intradermal, intramuscular and intravenous) administration, even though the best route of administration is dependent on the patient's status.

The formulations can be in the form of single doses. The formulations are prepared according to methods known in the field of pharmacology. The appropriate quantities of active substances suitable for administration may vary as a function of the particular field therapy. In general, the active substance concentration in a single-dose formulation is 5% to 95% of the total formulation.

The food compositions of the invention include any food composition to which compounds described in this invention have been added, or any food composition, which has been prepared in the presence of the compounds of the invention. Said compositions are preferably: milk shake, flavoured milk, milk, yoghurt, fermented milk, biscuit, juice, cake, bread, infant food, dehydrated food, oil, chewing gum, candies, or clinical nutrition formula.

The compounds of the invention can therefore be employed as nutritional supplements.

The invention provided by the application is illustrated by the non-limiting examples herein below.

### EXAMPLES

### Example 1

Cerebrospinal Fluid

Several rats were given 100 mg hydroxytyrosol by oral administration. After 15 min a small amount of cerebrospinal fluid was taken from the rat and analysed for the presence of hydroxytyrosol. Also the total antioxidant capacity in this fluid was measured and compared with a control. The detection of hydroxytyrosol was accomplished by gas chromatography and concentrations were determined by a standard curve. Hydroxytyrosol reached a concentration between 0.1 and 0.2 mM in cerebrospinal fluid after 15 minutes of oral administration. Moreover, the total antioxidant capacity of cerebrospinal fluid increased by 35% under these conditions as compared to a control.

### Example 2

Neuroprotection

The capacity of two phenolic compounds to rescue neuronal cells from different kind of stressors was tested. To primary hippocampal cultures from rat, different neurotoxins (glutamate, hydrogen peroxide or beta-amyloid peptide) were added in such concentrations that in half of the cells apoptosis was induced. Then, together with the toxins, hydroxytyrosol or tyrosol were added in a final concentration of 50 μM, and the cells were incubated for 24 h. at 37° C. Subsequently, apoptosis was measured in the cell cultures and the number of cells that were protected by this treatment as compared to a control was calculated. Both phenolic compounds tested protected part of the cells from dying, although hydroxytyrosol protected more cells than tyrosol did. Hydroxytyrosol protected around 60% of the cells against hydrogen peroxide, 35% against glutamate-induced neurotoxicity, and 45% against beta-amyloid induced apoptosis.

### Example 3

Prevention of Beta-Amyloid Precipitation

We found that hydroxytyrosol is able to bind iron, zinc and copper ions with high affinity. Therefore, an experiment was performed to study its capacity to solubilize beta-amyloid peptide from insoluble aggregates. Beta-amyloid peptide (1-40) was brought to 10 μM in 150 mM NaCl, 50 mM HEPES (pH 7.4), and mixed with 10 μM CuCl₂ or 10 μM FeCl₃. Precipitation of beta-amyloid peptide by copper and iron was measured by a turbidometric assay (Huang et al. J. Biol. Chem. 1997, 272(42), 26464-70). Copper- and iron-induced turbidity was almost completely abolished (>90%) by the presence of 25 μM hydroxytyrosol.

### Example 4

Monoamine Oxidase Inhibition by Hydroxytyrosol

We determined the IC50 of hydroxytyrosol for monoamine oxidase B (MAO-B). Therefore, MAO-B enzyme activities were measured using purified mitochondria from rat brain tissue as described previously (Holt et al. Anal. Biochem. 1997, 244, 384-392). Benzylamine (300 μM) was used as a substrate for MAO-B and the IC50 was calculated by adding different amounts of hydroxytyrosol to the enzyme assay. It was found that hydroxytyrosol is a strong inhibitor of MAO-B with an IC50 of 16.3±1.3 μM.

### Example 5

Absorption of Hydroxytyrosol and Derivatives

The absorption of hydroxytyrosol (II), and the ability of 2-(3,4-dihydroxyphenyl) ethyl acetate (V) and 2-(3,4-distearyloxyphenyl) ethyl stearate (XXII) to liberate hydroxytyrosol were studied in mice. Equivalent amounts of each compound (10 mg hydroxytyrosol, 13 mg 2-(3,4-dihydroxyphenyl) ethyl acetate and 70 mg 2-(3,4-distearyloxyphenyl) ethyl stearate) were given to mice by oral administration, and after 15 and 60 min. blood was collected from each animal. Plasma from each blood sample was subsequently extracted with ethyl acetate, and subjected to analysis by gas chromatography in combination with mass spectrometry. Both at 15 and 60 min after 2-(3,4-dihydroxyphenyl) ethyl acetate administration, the amount of hydroxytyrosol was very similar in plasma when compared to hydroxytyrosol administration (see table below).

| Compound | hydroxytyrosol in | Hydroxytyrosol in |
|---|---|---|
| administered | plasma, 15 min. | plasma, 60 min. |
| Hydroxytyrosol | 0.79 mg/L | 0.05 mg/L |
| 2-(3,4-dihydroxyphenyl) | 0.68 mg/L | 0.06 mg/L |
| ethyl acetate | | |
| 2-(3,4-distearyloxyphenyl) | not detected | 0.44 mg/L |
| ethyl stearate | | |

When 2-(3,4-distearyloxyphenyl) ethyl stearate was given to mice, hydroxytyrosol was only detected in plasma one hour after administration. After 15 min. no hydroxytyrosol could be detected in plasma samples (see table above). These results indicate that, although at different rates, both 2-(3,4-dihydroxyphenyl) ethyl acetate and 2-(3,4-distearyloxyphenyl) ethyl stearate liberate hydroxytyrosol in the stomach or intestine of mice which can subsequently be taken up an pass to the blood stream.

### Example 6

Preparation of an Enriched Juice

An enriched juice was prepared using the following ingredients:

| Ingredient | Amount per liter | |
|---|---|---|
| Concentrated juice | 200 | g |
| Tyrosol | 1 | g |
| Insoluble fibre | 10 | g |
| Lecithin | 0.5 | g |
| Flavors | 2 | g |
| Vitamin C | 90 | mg |
| Vitamin B1 | 2.1 | mg |
| Vitamin B2 | 2.4 | mg |
| Vitamin B6 | 3 | mg |
| Vitamin B12 | 1.5 | μg |
| Vitamin A | 1.2 | mg |
| Vitamin D | 7.5 | μg |
| Folic acid | 300 | μg |

Processing Technology

The final product was prepared from a concentrated juice by addition of water and water soluble ingredients. Then, tyrosol was added and mixed and the resulting product was pasteurised and homogenized. Finally, the product was cooled and packaged.

### Example 7

Preparation of an U.H.T Milk Based Product

A milk based product was prepared using the following ingredients:

| Ingredient | Amount per liter | |
|---|---|---|
| Skimmed milk | 960 | g |
| Skimmed milk powder | 17 | g |
| Hydroxytyrosol | 1 | g |
| Bisodium phosphate | 0.5 | g |
| Tripotasium phosphate | 0.2 | g |
| Water | 2 | g |
| Vitamin B6 | 3 | mg |
| Vitamin B12 | 3.8 | μg |
| Vitamin A | 1200 | μg |
| Vitamin D | 7.5 | μg |
| Vitamin E | 15 | mg |
| Folic acid | 300 | μg |

Processing Technology

All solid ingredients were mixed with the liquid milk and water. Then, hydroxytyrosol was admixed and homogenised in the absence of oxygen. The resulting dairy product was then subjected to U.H.T. treatment (150° C. for 4 to 6 seconds) and finally packaged in the absence of oxygen.

### Example 8

Preparation of a Liquid Nutritionally Balanced Diet Formulation

A liquid nutritionally balanced diet formula was prepared using the following ingredients:

| Ingredient | Amount per liter | g |
|---|---|---|
| Skimmed milk | 814.3 | g |
| Whey protein concentrate | 11.8 | g |
| Oil blend | 32.5 | g |
| Sacarose | 27 | g |
| Maltodextrine | 71 | g |
| Soluble Fibre | 10 | g |
| Vitamin A | 1200 | mg |
| Vitamin D | 7.5 | μg |
| Vitamin E | 15 | mg |
| Vitamin K | 120 | μg |
| Vitamin C | 90 | mg |
| Thiamine | 2.25 | mg |
| Riboflavin | 2.55 | mg |
| Pyridoxine | 3 | mg |
| Vitamin B12 | 3 | μg |
| Niacin | 28.5 | mg |
| Folic acid | 30 | μg |
| Panthotenic acid | 4 | mg |
| Biotin | 40 | μg |
| Calcium | 1200 | mg |
| Phosphorus | 970 | mg |
| Magnesium | 47 | mg |
| Sodium | 480 | mg |
| Potassium | 1000 | mg |
| Chloride | 700 | mg |
| Iron | 43 | mg |
| Zinc | 24.5 | mg |
| Iodine | 150 | μg |
| Manganese | 100 | μg |
| Selenium | 10 | μg |
| Mono- and diglycerides | 1.5 | g |
| Bisodium phosphate (Na₂HPO₄ 5H₂O) | 0.6 | g |
| Carrageenan | 4 | g |
| Demineralised Water | 70 | g |
| Flavor | 2.75 | g |
| 2-(3,4-dihydroxyphenyl) ethyl docosahexaenoate | 1.5 | g |

Processing Technology

To an appropriately sized blend tank with agitation and heating all solid ingredients were mixed with the liquid milk and water. Then, 2-(3,4-dihydroxyphenyl) ethyl docosahexaenoate was admixed. The mixture was then heated at 60-70° C. and emulsified through a single stage homogenizer at 6 to 7 MPa in absence of oxygen. After emulsification the mixture was heated to 140-150° C., 4-6 s, and was then passed through a two stages homogenizer (27-29 MPa and 3-4 MPa). Finally the mixture was packaged in absence of oxygen.

### Example 9

Synthesis of 2-(3.4-dihydroxyphenyl) Ethyl Acetate (V)

Powdered anhydrous K₂CO₃ (90 mg, 0.65 mmol), acetyl chloride (0.46 ml, 0.66 mmol) and tetrabutylammonium hydrogensulfate (TBAH, 22 mg, 0.06 mmol) were added to a stirred solution of 2-(3,4-dihydroxyphenyl) ethanol (100 mg, 0.65 mmol) in dry THF (5 ml). The mixture was stirred under argon at room temperature for 15 h. Then, the reaction mixture was filtered and evaporated to dryness. The residue was dissolved in dichloromethane (50 ml), washed with water (2×50 ml) and the organic layer was dried over anhydrous sodium sulphate and evaporated to dryness. The residue was purified by column chromatography using a solvent mixture of n-hexane: diethyl ether (1:1) to give 72 mg of a clear syrup in 57% yield.

¹H-NMR (300 mHz, CDCl₃): 6.78 (d, J=8.1 Hz, 1H, aromatic), 6.73 (d, J=1.5 Hz, 1H, aromatic), 6.63 (dd, J=8.0, 1.5 Hz, 1H, aromatic), 4.23 (t, J=7.1 Hz, 2H, —CH₂OOC—), 2.81 (t, J=7.1 Hz, 2H, ar-CH₂—), 2.03 (s, 3H, —CH₃).

### Example 10

Synthesis of 2-(3,4-Dihydroxyphenyl) Ethyl Stearate (VII)

Powdered anhydrous K₂CO₃ (90 mg, 0.65 mmol), stearoyl chloride (197 mg, 0.66 mmol) and 22 mg tetrabutylammonium hydrogensulfate (TBAH) were added to a stirred solution of 2-(3,4-dihydroxyphenyl) ethanol (100 mg, 0.65 mmol) in dry THF (5 ml). The mixture was stirred under argon at room temperature for 24 h. Then, the reaction mixture was filtrated and evaporated to dryness, solved in dichloromethane (50 ml) and washed with water (2×50 ml). The organic layer was dried over anhydrous sodium sulphate and evaporated to dryness. The residue was purified by column chromatography using a solvent mixture of n-hexane: diethyl ether (2:1) to give 132 mg of a white solid in 48% yield.

¹H-NMR (300 mHz, CDCl₃): 6.78 (d, J=8.1 Hz, 1H, aromatic), 6.72 (d, J=2, 1H, aromatic), 6.63 (dd, J=8.0, 2.0 Hz, 1H, aromatic), 5.34 (m, 2H, HC═CH), 4.22 (t, J=7.1 Hz, 2H, —CH₂OOC—), 2.81 (t, J=7.1 Hz, 2H, ar-CH₂—), 2.27 (t, J=7.4 Hz, 2H, —OOC—CH₂—), 1.58 (m, 2H, —OOC—CH₂—CH₂—), 1.24 (m, 28H, —CH₂—), 0.87 (t, J=6.9, 3H, —CH₃).

### Example 11

Synthesis of 2-(3,4-dihydroxyphenyl) Ethyl Oleate (XI)

Powdered anhydrous K₂CO₃ (90 mg, 0.65 mmol), oleoyl chloride (0.27 ml, 0.75 mmol) and tetrabutylammonium hydrogensulfate (TBAH) were added to a stirred solution of 2-(3,4-dihydroxyphenyl) ethanol (100 mg, 0.65 mmol) in dry THF (5 ml). The mixture was stirred under argon at room temperature for 24 h. Then, the reaction mixture was filtrated and evaporated to dryness, dissolved in dichloromethane (50 ml) and washed with water (2×50 ml). The organic layer was dried over anhydrous sodium sulphate and evaporated to dryness. The residue was purified by column chromatography using a solvent mixture of n-hexane: diethyl ether (4:1) to give 128 mg of lightly yellow syrup in 47% yield.

¹H-NMR (300 mHz, CDCl₃): 6.78 (d, J=8.1 Hz, 1H, aromatic), 6.72 (d, J=2, 1H, aromatic), 6.63(dd, J=8.0, 2.0 Hz, 1H, aromatic), 5.34 (m, 2H, HC═CH), 4.23 (t, J=7.1 Hz, 2H, —CH₂OOC—), 2.80 (t, J=7.1 Hz, 2H, ar-CH₂—), 2.28 (t, J=7.6 Hz, 2H, —OOC—CH₂—), 1.99 (m, 4H, —CH₂—HCCH—CH₂—), 1.58 (m, 2H, —OOC—CH₂—CH₂—), 1.26 (m, 26H, —CH₂—), 0.87 (t, J=6.9, 3H, —CH₃).

### Example 12

Synthesis of 2-(3,4-diacetoxyphenyl) Ethyl Acetate (XV)

To a solution of 2-(3,4-dihydroxyphenyl) ethanol (200 mg, 1.3 mmol) in dry THF (10 ml), pyridine (0.5 ml), acetic anhydride (0.6 ml), and 4-dimethylaminopyridine (30 mg) were added. The mixture was stirred under argon at room temperature for 7 h. Then, methanol (25 ml) was added and the mixture was co-evaporated with toluene (3×10 ml) to dryness. The crude was purified by column chromatography using a solvent mixture of n-hexane: diethyl ether (1:1) to give 136 mg of syrup in 75% yield.

¹H-NMR (300 mHz, CDCl₃): 7.10 (dd, J=10.2, 1.9 Hz, 2H, aromatic), 7.04 (s, 1H, aromatic), 4.26 (t, J=6.9 Hz, 2H, —CH₂OOC—), 2.89 (t, J=6.9 Hz, 2H ar-CH₂—), 2.27 (s, 3H, -ar-OCOCH₃), 2.26 (s, 3H, -ar-OCOCH₃), 2.02 (s, 3H, CH₃).

### Example 13

Synthesis of 2-(3.4-distearoylphenyl) Ethyl Stearate (XVII)

To a stirred solution of 2-(3,4-dihydroxyphenyl) ethanol (100 mg, 0.65 mmol) in dry THF (10 ml), stearic acid (563 mg, 1.98 mmol), dicyclohexylcarbodiimide (410 mg, 1.98 mmol), and 4-dimethylaminopyridine (25 mg, 0.19 mmol) were added at 0° C. The mixture was stirred under argon at room temperature for 24 h. Precipitated urea was then filtered off and the filtrate evaporated to dryness. The residue was dissolved in dichloromethane (25 ml) and washed twice with 0.5 N HCl (2×50 ml), with saturated NaHCO₃ solution, and brine (1×50 ml), and finally dried over anhydrous sodium sulphate and evaporated to dryness. The residue was purified by column chromatography using a solvent mixture of n-hexane: diethyl ether (6:1) to give 200 mg (32%) of a white solid.

¹H-NMR (300 mHz, CDCl₃): 7.08 (m, AB system, 2H aromatic), 7.02 (s, 1H, aromatic), 4.26 (t, J=7.0 Hz, —CH₂OOC—), 2.91 (t, J=7.0 Hz, 2H ar-CH₂—), 2.50 (t, J=7.5, Hz 4H ar-OOC—CH₂—), 2.26 (t, J=7.5 Hz, 2H, —OOC—CH₂—), 1.71 (m, 4H, -ar-OOC—CH₂—CH₂—),1.62 (m, 2H, —OOC—CH₂—CH₂—), 1.24 (m, 84H —CH₂—), 0.87 (t, J=6.9, 9H, —CH₃).

1. A phenolic compound of the formula 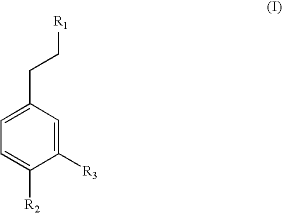
wherein R₁ and R₂ are independently selected from: OH, OCOalkyl or OCOalkenyl, and R₃ is either H, OH, OCOalkyl or OCOalkenyl, and wherein the alkyl or alkenyl group contains from 2 to 22 carbon atoms.

2. A phenolic compound according to claim 1 selected from the group consisting of hydroxytyrosol, tyrosol and oleuropein according to formulas II, III and IV, respectively.

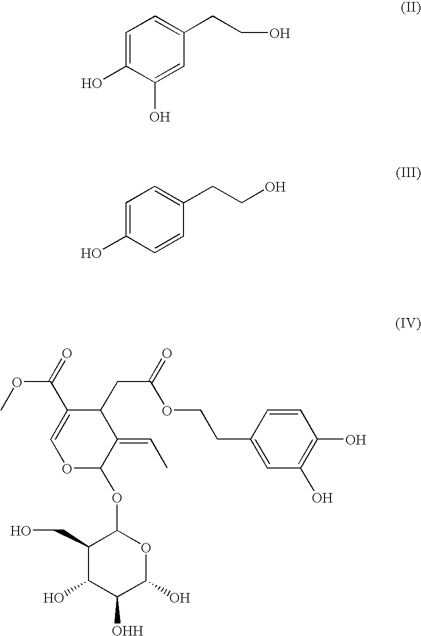

3. A phenolic compound according to claim 1, which liberates hydroxytyrosol or tyrosol after hydrolysis, and which is selected from the group consisting of 2-(3,4-dihydroxyphenyl) ethyl acetate (V), 2-(3,4-dihydroxyphenyl) ethyl oleate (VI), 2-(3,4-dihydroxyphenyl) ethyl stearate (VII), 2-(3,4-dihydroxyphenyl) ethyl docosahexaenoate (VIII), 2-(3,4-dihydroxyphenyl) ethyl eicosapentaenoate (IX), 2-(4-hydroxyphenyl) ethyl acetate (X), 2-(4-hydroxyphenyl) ethyl oleate (XI), 2-(4-hydroxyphenyl) ethyl stearate (XII), 2-(4-hydroxyphenyl) ethyl docosahexaenoate (XIII), 2-(4-hydroxyphenyl) ethyl eicosapentaenoate (XIV), 2-(3,4-diacetoxyphenyl) ethyl acetate (XV), 2-(3,4-dioleyloxyphenyl) ethyl oleate (XVI), 2-(3,4-distearyloxyphenyl) ethyl stearate (XVII), 2-(3,4-didocosahexaenoyloxyphenyl) ethyl docosahexaenoate (XVIII), 2-(3,4-dieicosapentaenoyloxyphenyl) ethyl eicosapentaenoate (XIX), 2-(4-acetoxyphenyl) ethyl acetate (XX), 2-(4-oleyloxyphenyl) ethyl oleate (XXI), 2-(4-stearyloxyphenyl) ethyl stearate (XXII), 2-(4-docosahexaenoyloxyphenyl) ethyl docosahexaenoate (XXIII), and 2-(4-eicosapentaenoyloxyphenyl) ethyl eicosapentaenoate (XXIV).

4. A compound according to claim 1 selected from the group consisting of
2-(3,4-dihydroxyphenyl) ethyl oleate (VI),
2-(3,4-dihydroxyphenyl) ethyl stearate (VII),
2-(3,4-dihydroxyphenyl) ethyl docosahexaenoate (VIII),
2-(3,4-dihydroxyphenyl) ethyl eicosapentaenoate (IX),
2-(4-hydroxyphenyl) ethyl docosahexaenoate (XIII),
2-(4-hydroxyphenyl) ethyl eicosapentaenoate (XIV),
2-(3,4-dioleyloxyphenyl) ethyl oleate (XVI),
2-(3,4-distearyloxyphenyl) ethyl stearate (XVII),
(3,4-didocosahexaenoyloxyphenyl) ethyl docosahexaenoate (XVIII),
2-(3,4-dieicosapentaenoyloxyphenyl) ethyl eicosapentaenoate (XIX),
2-(4-oleyloxyphenyl) ethyl oleate (XXI),
2-(4-stearyloxyphenyl) ethyl stearate (XXII),
2-(4-docosahexaenoyloxyphenyl) ethyl docosahexaenoate (XXIII), and
2-(4-eicosapentaenoyloxyphenyl) ethyl eicosapentaenoate (XXIV).

5. A pharmaceutical composition comprising a therapeutically effective amount of a compound according to claim 1 in a pharmaceutically acceptable carrier.

6. A pharmaceutical composition according to claim 5 formulated for oral administration to a patient.

7. A pharmaceutical composition according to claim 5 formulated for parenteral administration to a patient.

8. A food composition to which a therapeutically effective amount of a compound in accordance with claim 1 has been added.

9. The food composition of claim 8 which is selected from the group consisting of a milk shake, a flavoured milk, a milk, a yoghurt, a fermented milk, a biscuit, a juice, a cake, a bread, an infant food, a dehydrated food, an oil, a chewing gum, a candy, a clinical nutrition formula, and a nutritional supplement.

10. The use of a phenolic compound of the formula 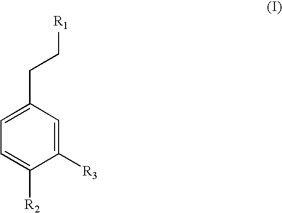
wherein R₁ and R₂ are independently selected from: OH, OCOalkyl or OCOalkenyl, and R₃ is either H, OH, OCOalkyl or OCOalkenyl, and wherein the alkyl or alkenyl group contains from 2 to 22 carbon atoms for the treatment or prevention of a neurodegenerative disease selected from the group consisting of Creutzfeldt-Jakob disease, Alzheimer's disease, Huntington's disease, Lewy body disease, Parkinson's disease, Pick's disease, amyotrophic lateral sclerosis, neurofibromatosis, brain injury, stroke, multiple sclerosis, loss of memory, and multiple infarct dementia.

11. The use according to claim 10 where the phenolic compound is selected from the group consisting of hydroxytyrosol, tyrosol and oleuropein according to formulas II, III and IV, respectively.

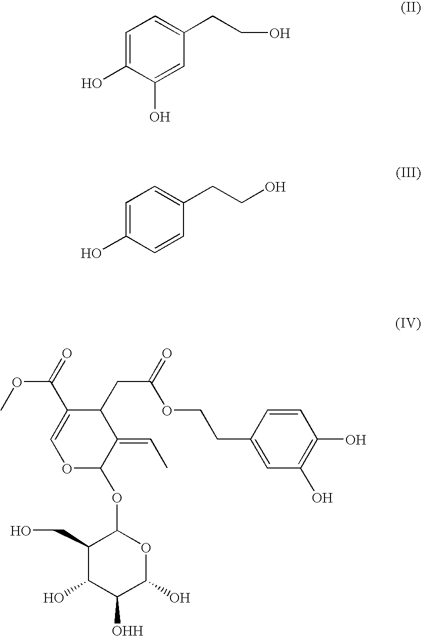

12. The use according to claim 10 where the phenolic compound liberates hydroxytyrosol or tyrosol after hydrolysis, and which is selected from the group consisting of 2-(3,4-dihydroxyphenyl) ethyl acetate (V), 2-(3,4-dihydroxyphenyl) ethyl oleate (VI), 2-(3,4-dihydroxyphenyl) ethyl stearate (VII), 2-(3,4-dihydroxyphenyl) ethyl docosahexaenoate (VIII), 2-(3,4-dihydroxyphenyl) ethyl eicosapentaenoate (IX), 2-(4-hydroxyphenyl) ethyl acetate (X), 2-(4-hydroxyphenyl) ethyl oleate (XI), 2-(4-hydroxyphenyl) ethyl stearate (XII), 2-(4-hydroxyphenyl) ethyl docosahexaenoate (XIII), 2-(4-hydroxyphenyl) ethyl eicosapentaenoate (XIV), 2-(3,4-diacetoxyphenyl) ethyl acetate (XV), 2-(3,4-dioleyloxyphenyl) ethyl oleate (XVI), 2-(3,4-distearyloxyphenyl) ethyl stearate (XVII), 2-(3,4-didocosahexaenoyloxyphenyl) ethyl docosahexaenoate (XVIII), 2-(3,4-dieicosapentaenoyloxyphenyl) ethyl eicosapentaenoate (XIX), 2-(4-acetoxyphenyl) ethyl acetate (XX), 2-(4-oleyloxyphenyl) ethyl oleate (XXI), 2-(4-stearyloxyphenyl) ethyl stearate (XXII), 2-(4-docosahexaenoyloxyphenyl) ethyl docosahexaenoate (XXIII), and 2-(4-eicosapentaenoyloxyphenyl) ethyl eicosapentaenoate (XXIV).

13. The use according to claim 10 where the phenolic compound is selected from the group consisting of
2-(3,4-dihydroxyphenyl) ethyl oleate (VI),
2-(3,4-dihydroxyphenyl) ethyl stearate (VII),
2-(3,4-dihydroxyphenyl) ethyl docosahexaenoate (VIII),
2-(3,4-dihydroxyphenyl) ethyl eicosapentaenoate (IX),
2-(4-hydroxyphenyl) ethyl docosahexaenoate (XIII),
2-(4-hydroxyphenyl) ethyl eicosapentaenoate (XIV),
2-(3,4-dioleyloxyphenyl) ethyl oleate (XVI),
2-(3,4-distearyloxyphenyl) ethyl stearate (XVII),
(3,4-didocosahexaenoyloxyphenyl) ethyl docosahexaenoate (XVIII),
2-(3,4-dieicosapentaenoyloxyphenyl) ethyl eicosapentaenoate (XIX),
2-(4-oleyloxyphenyl) ethyl oleate (XXI),
2-(4-stearyloxyphenyl) ethyl stearate (XXII),
2-(4-docosahexaenoyloxyphenyl) ethyl docosahexaenoate (XXIII), and
2-(4-eicosapentaenoyloxyphenyl) ethyl eicosapentaenoate (XXIV).
